(19) United States
(12) Patent Application Publication
(10) Pub. No.: US 2024/0277509 A1
(43) Pub. Date: Aug. 22, 2024

(54) ARM IMMOBILIZERS AND RELATED METHODS

(71) Applicant: Brian Christopher Lynch, Durham, NC (US)

(72) Inventor: Brian Christopher Lynch, Durham, NC (US)

(21) Appl. No.: 18/441,149

(22) Filed: Feb. 14, 2024

Related U.S. Application Data
(60) Provisional application No. 63/446,931, filed on Feb. 20, 2023.

(51) Int. Cl. A61F 5/37

(52) U.S. Cl. CPC A61F 5/3723

(57) ABSTRACT

Arm immobilizers and related methods are disclosed. According to an aspect, an arm immobilizer includes an elongated, flexible material having a first end and a second end. Further, the arm immobilizer includes a first part of a hook and fastener material attached to the first end. The arm immobilizer includes a second part of the hook and fastener material being attached to a portion of the elongated, flexible material positioned at a predefined location between the first end and the second end. The arm immobilizer includes a hoop shaped component that defines an aperture and is attached to the second end. The hoop shaped component is sized for receiving the first end through the aperture such that the first end is foldable towards the predefined location between the first end and the second end for attachment of the first part to the second part of the hook and fastener material.

### CROSS REFERENCE TO RELATED APPLICATION

This application claims priority to U.S. Provisional Patent Application No. 63/446,931, filed Feb. 20, 2023, and titled ARM IMMOBILIZERS AND RELATED METHODS, the content of which is incorporated by reference herein in its entirety.

### TECHNICAL FIELD

The presently disclosed subject matter relates generally to healthcare and medical equipment. Particularly, the presently disclosed subject matter relates to arm immobilizers and related methods.

### BACKGROUND

Arm slings and various other arm immobilizers have been used to restrict or limit arm movement to allow the arm or shoulder to heal and also to prevent further injury. Generally, an arm sling includes a portion that carries or otherwise holds the forearm in a set position, and another portion that wraps around the user's neck for securing it to the user.

Currently available arm slings can be difficult for the wearer to put on. In most cases, the assistance of another person is needed to help the wearer to put on the arm sling. This can be inconvenient. Therefore, it is desired to provide arm slings and other arm immobilizers that the wearer can independently put on.

Further, currently available arm slings can cause stiffness in the elbow, hand, digits, and wrist when worn for an extended period of time. Therefore, it is desired to provide improved arm slings and other arm immobilizers that avoid or reduce such stiffness by allowing movement. This will also allow the wearer additional independence in the ability to use both hands.

### BRIEF DESCRIPTION OF THE DRAWINGS

Having thus described the presently disclosed subject matter in general terms, reference will now be made to the accompanying Drawings, which are not necessarily drawn to scale, and wherein:

FIG. 1 is a top view of an arm immobilizer in a closed configuration for wear in accordance with embodiments of the present disclosure;

FIG. 2 is another top view of the arm immobilizer shown in FIG. 1 in the closed configuration with the end not folded over for attachment of the hook and fastener material;

FIG. 3 is a top view of the arm immobilizer shown in FIGS. 1 and 2 in an open configuration;

FIG. 4 is a bottom view of the arm immobilizer shown in FIGS. 1-3 in an open configuration showing a side that opposes the side shown in FIG. 3;

FIG. 5 is a front view of the arm immobilizer in an initial configuration for being put on a user in accordance with embodiments of the present disclosure;

FIG. 6 is a front view showing of a step subsequent to the initial configured shown in FIG. 5 in which the hook and fastener material is attached for securing the arm immobilizer about the upper part of the right arm and the torso;

FIG. 7 is a front view showing a final configuration of the arm immobilizer for securing the upper part of the right arm to the torso;

FIG. 8 is a front view showing the user removing the arm immobilizer;

FIG. 9 is a front view showing the arm immobilizer encircling a right arm and torso of the user in a step subsequent to the step shown in FIG. 8;

FIG. 10 is a front view of a subsequent step in which the hook and fastener material is attached for securing the arm immobilizer about the upper part of the right arm and the torso;

FIG. 11 is a front view showing a final configuration of the arm immobilizer for securing the upper part of the right arm to the torso; and

FIG. 12 is a front view showing the user removing the arm immobilizer.

### SUMMARY

The presently disclosed subject matter relates to arm immobilizers and related methods. According to an aspect, an arm immobilizer includes an elongated, flexible material having a first end and a second end. Further, the arm immobilizer includes a first part of a hook and fastener material being attached to the first end. The arm immobilizer also includes a second part of the hook and fastener material being attached to a portion of the elongated, flexible material positioned at a predefined location between the first end and the second end. The arm immobilizer also includes a hoop shaped component that defines an aperture and is attached to the second end. The hoop shaped component is sized for receiving the first end through the aperture such that the first end is foldable towards the predefined location between the first end and the second end for attachment of the first part of the hook and fastener material to the second part of the hook and fastener material.

According to another aspect, a method of using an arm immobilizer includes providing an arm immobilizer having an elongated, flexible material having a first end and a second end. The arm immobilizer also includes a first part of a hook and fastener material being attached to the first end. Further, the arm immobilizer includes a second part of the hook and fastener material being attached to a portion of the elongated, flexible material positioned at a predefined location between the first end and the second end. The arm immobilizer also includes a hoop shaped component that defines an aperture and is attached to the second end, foldable towards the predefined location between the first end and the second end for attachment of the first part of the hook and fastener material to the second part of the hook and fastener material. The method also includes inserting the first end through the aperture of the hoop. Further, the method includes using a second arm of the user to move the elongated, flexible material to a position that encircles a torso and first arm of a user. The method also includes using the second arm of the user to fold the first end towards the predefined location between the first end and the second end for attachment of the first part of the hook and fastener material to the second part of the hook and fastener material such that at least a portion of the first arm of the user is substantially immobilized.

### DETAILED DESCRIPTION

The following detailed description is made with reference to the figures. Exemplary embodiments are described to illustrate the disclosure, not to limit its scope, which is defined by the claims. Those of ordinary skill in the art will recognize a number of equivalent variations in the description that follows.

Articles “a” and “an” are used herein to refer to one or to more than one (i.e. at least one) of the grammatical object of the article. By way of example, “an element” means at least one element and can include more than one element.

“About” is used to provide flexibility to a numerical endpoint by providing that a given value may be “slightly above” or “slightly below” the endpoint without affecting the desired result.

The use herein of the terms “including,” “comprising,” or “having,” and variations thereof is meant to encompass the elements listed thereafter and equivalents thereof as well as additional elements. Embodiments recited as “including,” “comprising,” or “having” certain elements are also contemplated as “consisting essentially of” and “consisting” of those certain elements.

Recitation of ranges of values herein are merely intended to serve as a shorthand method of referring individually to each separate value falling within the range, unless otherwise indicated herein, and each separate value is incorporated into the specification as if it were individually recited herein. For example, if a range is stated as between 1%-50%, it is intended that values such as between 2%-40%, 10%-30%, or 1%-3%, etc. are expressly enumerated in this specification. These are only examples of what is specifically intended, and all possible combinations of numerical values between and including the lowest value and the highest value enumerated are to be considered to be expressly stated in this disclosure.

Unless otherwise defined, all technical terms used herein have the same meaning as commonly understood by one of ordinary skill in the art to which this disclosure belongs.

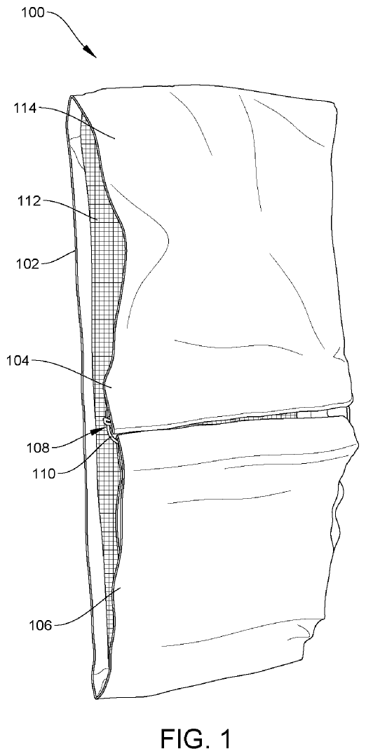

FIG. 1 is top view of an arm immobilizer 100 in a closed configuration for wear in accordance with embodiments of the present disclosure. In particularly, in the wear configuration as shown in FIG. 1, the arm immobilizer 100 includes an elongated, flexible material 102 that is formed into a looped shape such that it can be fit around a torso and upper portion of the wearer's (or user's) arm (not shown). In this configuration, the arm immobilizer 100 can restrict or limit movement of the upper arm. The elongated, flexible material 102 includes two different ends, referred to as a first end 104 and a second end 106.

As shown in FIG. 1, the second end 106 is inserted through an aperture 108 of a hoop shaped component 110 that is attached to the first end 108. The folded-over portion of the end 106 has a hook and fastener material (not shown in FIG. 1) attached to it that is affixed to a mating hook and fastener material (not shown in FIG. 1) on the elongated, flexible material 102. This mating hook and fastener material is attached to the elongated, flexible material 102 at a predefined location between the first end 104 and the second end 106.

Regarding sizing, the parts/components of the arm immobilizer 100 can be suitably sized and shaped to fit differently sized and shaped people. For example, the arm immobilizer 100 may be smaller than shown to fit children or teenagers. Also, for example, it is also noted that the hook and fastener material may be placed differently and shaped differently to accommodate differently sized people. For example, the hook and fastener material may be larger or smaller in size, or placed at a different located between the first end 104 and the second end 106.

The hoop shaped component 110 is a looped metal wire in this example. However, the component 110 may alternatively be made of different metals, plastic, or wood.

With continuing reference to FIG. 1, the elongated, flexible material 102 is substantially rectangular in shape in this example, although it may alternatively be any other suitable shape. The elongated, flexible material 102 has multiple layers in this example, but may alternatively be a single layer of material. The layers may be suitably attached together, such as by sewing or adhesive. In this example, the material 102 has two layers: a first layer 112 and a second layer 114. The first layer 112 has a higher coefficient of friction of friction than the second layer. Layer 114 can be any type of fabric or plastic. Layer 112 is a rubber-type material.

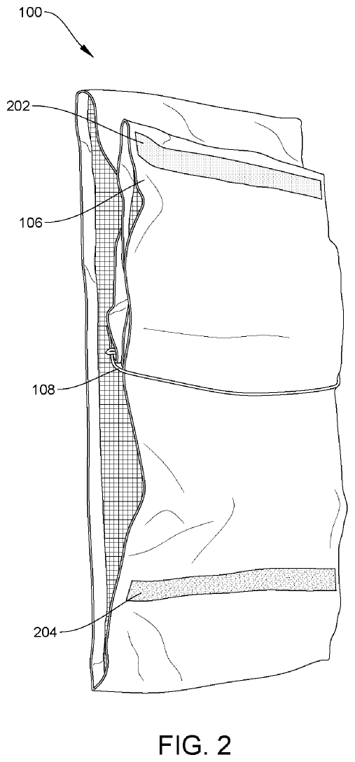

FIG. 2 is another top view of the arm immobilizer 100 shown in FIG. 1 in the closed configuration with the end 106 not folded over for attachment of the hook and fastener material. Referring to FIG. 2, a first part 200 and a second part 202 of a hook and fastener material are shown. The first part 200 and the second part 202 can be contacted together for attachment. An alternative could be buttons or metal fasteners.

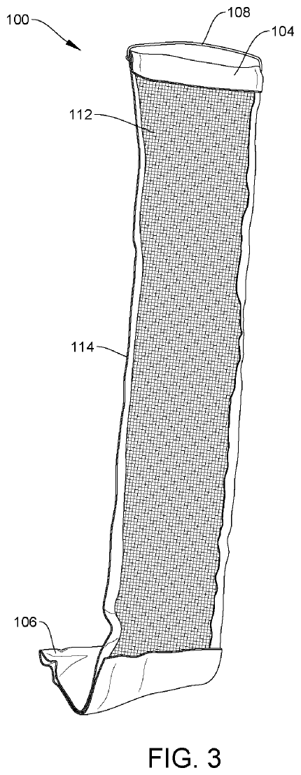

FIG. 3 is a top view of the arm immobilizer 100 shown in FIGS. 1 and 2 in an open configuration. Referring to FIG. 3, in this open configuration the elongated, flexible material 102 is not folded or looped. As shown, a side of the first layer 112 is shown. The exposed side of the first layer is rectangular in shape.

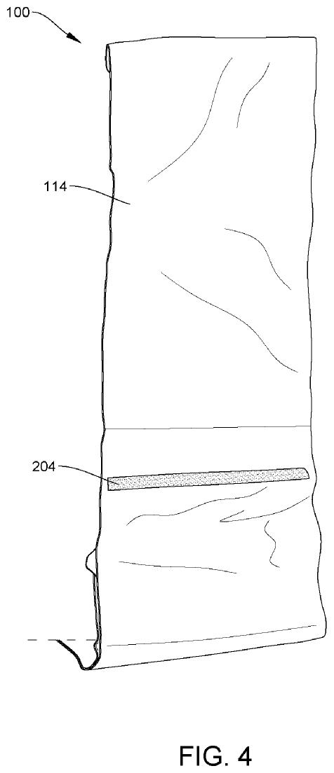

FIG. 4 is a bottom view of the arm immobilizer 100 shown in FIGS. 1- 3 in an open configuration showing a side that opposes the side shown in FIG. 3. Referring to FIG. 4, layer 114 is shown with the second part 202 of the hook and fastener material being attached thereto.

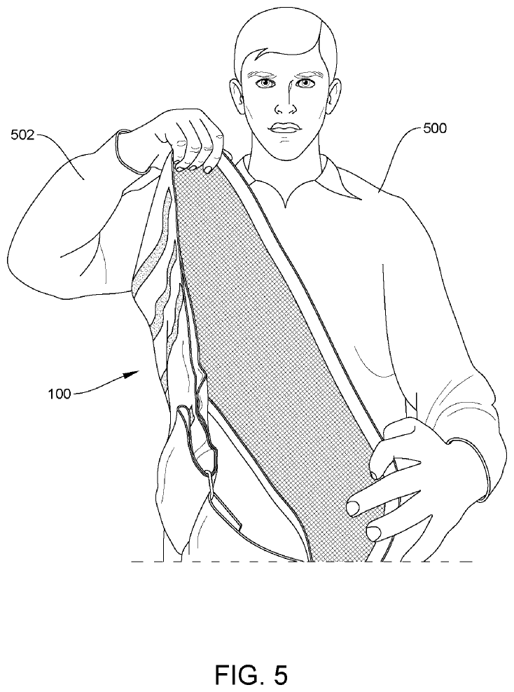

FIG. 5 is a front view of the arm immobilizer 100 in an initial configuration for being put on a user 500 in accordance with embodiments of the present disclosure. Referring to FIG. 5, the arm immobilizer 100 is being held by the user 500 in a looped configuration prior to being put on the user's right arm 502.

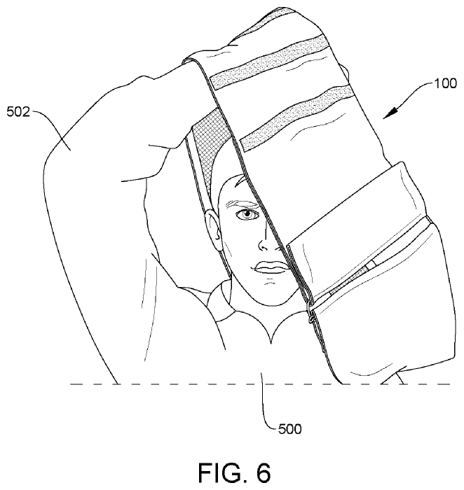

Now turning to FIG. 6, this figure shows the arm immobilizer 100 in a next step being raised over the user's 500 head.

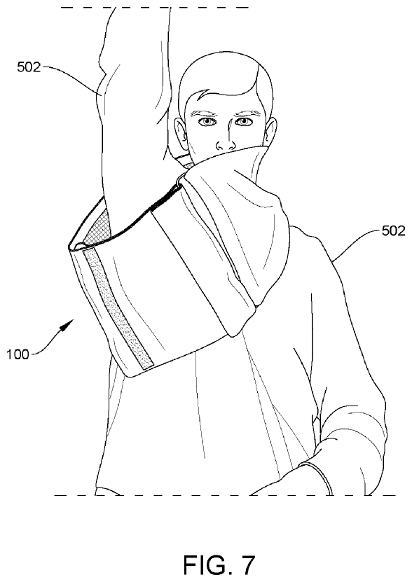

FIG. 7 is front view of the arm immobilizer 100 in a step following the step shown in FIG. 6. In this figure, the right arm 502 is placed through the arm immobilizer 100 as shown.

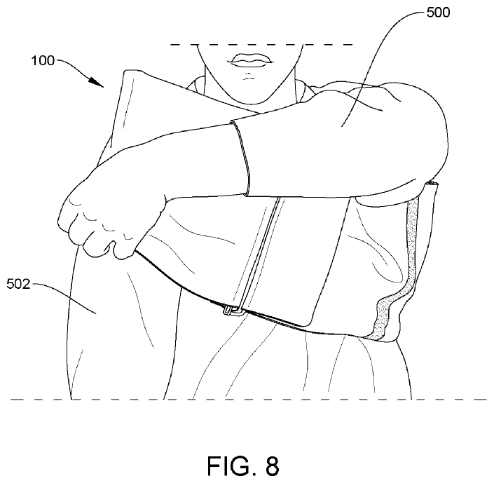

FIG. 8 is a front view of the arm immobilizer 100 in a step following the step shown in FIG. 7. In this figure, the arm immobilizer 100 is placed over the upper part of the arm 502 as shown.

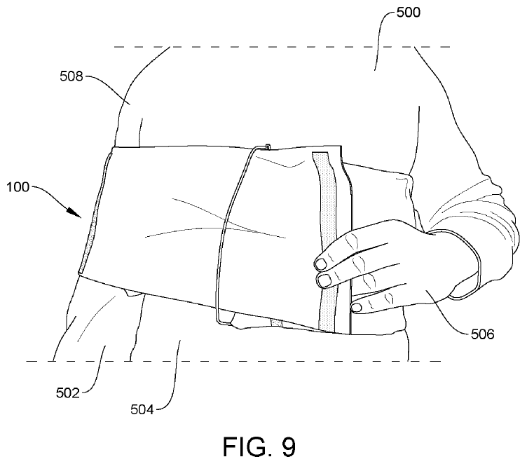

FIG. 9 is a front view showing the arm immobilizer 100 encircling a right arm 502 and torso 504 of the user 500 in a step subsequent to the step shown in FIG. 8. In this example, the right arm 502 is the injured arm and needs restriction of movement. The user's 500 left arm 506 is being used to position the arm immobilizer 100 as shown and to take the additional steps to suitably tighten the arm immobilizer 100 for securing the upper part of the right arm 502 against the torso 504. As a result, the upper part of the right arm 502 and right shoulder 508 have limited movement, but the lower part of the right arm 502 can be utilized to an extent.

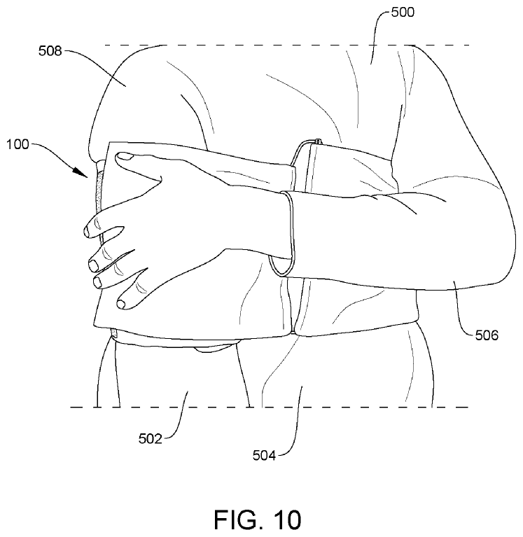

It is noted that in the configuration shown in FIG. 9 the end 106 is not folded over for the final configuration of securing the hook and fastener material. FIG. 10 is a front view of a subsequent step in which the hook and fastener material is attached for securing the arm immobilizer 100 about the upper part of the right arm 502 and the torso 504. In FIG. 10, it is shown that the left arm 506 is being used to press together the hook and fastener material.

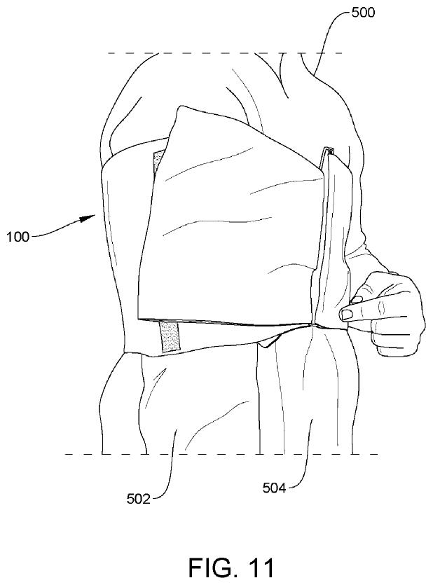

Now turning to FIG. 11, this figure is a front view showing a final configuration of the arm immobilizer 100 for securing the upper part of the right arm 502 to the torso 504.

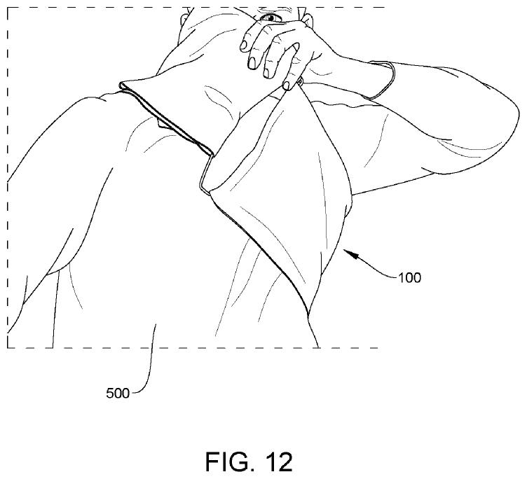

FIG. 12 is a front view showing the user 500 removing the arm immobilizer 100.

While the embodiments have been described in connection with the various embodiments of the various figures, it is to be understood that other similar embodiments may be used, or modifications and additions may be made to the described embodiment for performing the same function without deviating therefrom. Therefore, the disclosed embodiments should not be limited to any single embodiment, but rather should be construed in breadth and scope in accordance with the appended claims.

What is claimed is:

1. An arm immobilizer comprising:
an elongated, flexible material having a first end and a second end;
a first part of a hook and fastener material being attached to the first end;
a second part of the hook and fastener material being attached to a portion of the elongated, flexible material positioned at a predefined location between the first end and the second end; and
a hoop shaped component that defines an aperture and is attached to the second end, wherein the hoop shaped component is sized for receiving the first end through the aperture such that the first end is foldable towards the predefined location between the first end and the second end for attachment of the first part of the hook and fastener material to the second part of the hook and fastener material.

2. The arm immobilizer of claim 1, wherein the elongated, flexible material is substantially rectangular in shape.

3. The arm immobilizer of claim 1, wherein the second end defined an aperture for attachment of the hoop.

4. The arm immobilizer of claim 3, wherein the first end is folded upon and attached to itself to form the aperture for attachment of the hoop.

5. The arm immobilizer of claim 1, wherein the elongated, flexible material defines a first side and a second side that each extend between the first end and the second end.

6. The arm immobilizer of claim 5, wherein the first and second parts of the hook and fastener material extend substantially between the first side and the second side.

7. The arm immobilizer of claim 1, wherein the hook is made of a flexible material.

8. The arm immobilizer of claim 1, wherein the elongated, flexible material comprises multiple layers.

9. The arm immobilizer of claim 8, wherein the multiple layers comprise a first layer on one side and a second layer on an opposing side, and wherein the first layer has a higher coefficient of friction than the second layer.

10. The arm immobilizer of claim 9, wherein the second part of the hook and fastener material is attached to the second layer.

11. A method of using an arm immobilizer, the method comprising:
providing an arm immobilizer comprising:
an elongated, flexible material having a first end and a second end;
a first part of a hook and fastener material being attached to the first end;
a second part of the hook and fastener material being attached to a portion of the elongated, flexible material positioned at a predefined location between the first end and the second end;
a hoop shaped component that defines an aperture and is attached to the second end, foldable towards the predefined location between the first end and the second end for attachment of the first part of the hook and fastener material to the second part of the hook and fastener material;
inserting the first end through the aperture of the hoop;
using a second arm of the user to move the elongated, flexible material to a position that encircles a torso and first arm of a user; and
using the second arm of the user to fold the first end towards the predefined location between the first end and the second end for attachment of the first part of the hook and fastener material to the second part of the hook and fastener material such that at least a portion of the first arm of the user is substantially immobilized.

12. The method of claim 11, wherein the attachment of the first part of the hook and fastener material to the second part of the hook and fastener material at least substantially immobilizes an upper part of the first arm.

13. The method of claim 1, wherein the second end defined an aperture for attachment of the hoop.

14. The method of claim 3, wherein the first end is folded upon and attached to itself to form the aperture for attachment of the hoop.

15. The method of claim 1, wherein the elongated, flexible material defines a first side and a second side that each extend between the first end and the second end.

16. The method of claim 5, wherein the first and second parts of the hook and fastener material extend substantially between the first side and the second side.

17. The method of claim 1, wherein the hook is made of a flexible material.

18. The method of claim 1, wherein the elongated, flexible material comprises multiple layers.

19. The method of claim 8, wherein the multiple layers comprise a first layer on one side and a second layer on an opposing side, and wherein the first layer has a higher coefficient of friction than the second layer.

20. The method of claim 9, wherein the second part of the hook and fastener material is attached to the second layer.
